(19) United States
(12) Patent Application Publication
(10) Pub. No.: US 2020/0214264 A1
(43) Pub. Date: Jul. 9, 2020

(54) METHOD AND SYSTEM FOR MONITORING EPIDEMIC DISEASE IN PIGGERY

(71) Applicant: Beijing Etag Technology Company, Ltd., Beijing (CN)

(72) Inventors: Xingfu Zhou, Beijing (CN); Jiping Ren, Beijing (CN); Chao Pang, Beijing (CN); Dongge Hu, Beijing (CN)

(21) Appl. No.: 16/590,761

(22) Filed: Oct. 2, 2019

(30) Foreign Application Priority Data
Jan. 8, 2019 (CN) 201910015002.0

(51) Int. Cl. A01K 29/00; G16H 50/80; G16H 50/30; G06Q 50/02; G16H 10/60

(52) U.S. Cl. CPC A01K 29/005; G16H 50/80; G16H 10/60; G06Q 50/02; G16H 50/30

(57) ABSTRACT

The present invention discloses a method and system for monitoring an epidemic disease in a piggery. The monitoring method includes: obtaining a historical food intake amount, historical environmental parameters, a historical health state and a historical epidemic disease state of a live pig, where the historical environmental parameters include a temperature, a humidity, and concentration of gas in the air; establishing a piggery monitoring model by taking the historical food intake amount and the historical environmental parameters as an input and the historical health state and the historical epidemic disease state as an output; determining a first current state of the live pig according to the piggery monitoring model, where the first current state includes a healthy state and an epidemic disease state; and monitoring a monitored piggery according to the first current state.

### RELATED APPLICATION

This application claims benefit of priority of China Patent Application No. 201910015002.0, filed Jan. 8, 2019, entitled: METHOD AND SYSTEM FOR MONITORING EPIDEMIC DISEASE IN PIGGERY. The above-identified, related application is incorporated herein by reference in its entirety.

### FIELD OF USE

The present invention relates to the field of monitoring of epidemic diseases in a piggery, and in particular to a method and system for monitoring an epidemic disease in a piggery.

### BACKGROUND OF THE INVENTION

Epidemic diseases in a piggery have a huge impact, but there hasn't been an effective solution to provide a breeding guarantee for a farmer till now and the early detection and early treatment cannot be achieved. Existing epidemic diseases in a piggery are monitored artificially. However, due to the fast spreading of the epidemic diseases in a piggery, it is too late for the detection of the farmer, thus causing an enormous loss for the farmer or even affecting an order of a nationwide economic market.

### SUMMARY OF THE INVENTION

To achieve the above objective, the present invention provides the following solution.

A method for monitoring an epidemic disease in a piggery includes:

obtaining a historical food intake amount, historical environmental parameters, a historical health state and a historical epidemic disease state of a live pig, where the historical environmental parameters include a temperature, a humidity, and concentration of gas in the air;

establishing a piggery monitoring model by taking the historical food intake amount and the historical environmental parameters as an input and the historical health state and the historical epidemic disease state as an output;

determining a first current state of the live pig according to the piggery monitoring model, where the first current state includes a healthy state and an epidemic disease state; and

monitoring a monitored piggery according to the first current state.

Optionally, the determining a first current state of the live pig according to the piggery monitoring model specifically includes:

obtaining a current food intake amount of the live pig within a period of time threshold range;

determining whether the current food intake amount is in a downtrend within the time threshold range to obtain a first determining result;

if the first determining result indicates that the current food intake amount is in the downtrend within the time threshold range, obtaining current environmental parameters of the monitored piggery;

determining whether the current environmental parameters and the historical environmental parameters are within an environmental-parameter similarity threshold range to obtain a second determining result; and

if the second determining result indicates that the current environmental parameters and the historical environmental parameters are within the environmental-parameter similarity threshold range, determining, by taking the current food intake amount and the current environmental parameters as an input, the first current state of the live pig according to the piggery monitoring model.

Optionally, after monitoring the monitored piggery according to the first current state, the method further includes:

obtaining a second current state of any live pig in the monitored piggery;

determining whether the second current state and the first current state are within a first current-state similarity threshold range to obtain a third determining result;

if the third determining result indicates that the second current state and the first current state are within the first current-state similarity threshold range, determining that no epidemic disease occurs in the monitored piggery; and

if the third determining result indicates that the second current state and the first current state are not within the first current-state similarity threshold range, determining that an epidemic disease occurs in the monitored piggery and making an alarm to a farmer.

Optionally, after determining that an epidemic disease occurs in the monitored piggery and making the alarm to the farmer, the method further includes:

obtaining a third current state of a live pig in any piggery;

determining whether the third current state and the first current state are within a second current-state similarity threshold range to obtain a fourth determining result;

if the third current state and the first current state are within the second current-state similarity threshold range, determining that the epidemic disease does not occur in the any piggery; and

if the third current state and the first current state are not within the second current-state similarity threshold range, determining that the epidemic disease occurs in the any piggery and making an alarm to the farmer.

A system for monitoring an epidemic disease in a piggery includes:

a parameter obtaining module, configured to obtain a historical food intake amount, historical environmental parameters, a historical health state and a historical epidemic disease state of a live pig, where the historical environmental parameters include a temperature, a humidity, and concentration of gas in the air;

a piggery monitoring model establishment module, configured to establish a piggery monitoring model by taking the historical food intake amount and the historical environmental parameters as an input and the historical health state and the historical epidemic disease state as an output;

a first current state determination module, configured to determine a first current state of the live pig according to the piggery monitoring model, where the first current state includes a healthy state and an epidemic disease state; and

a monitoring module, configured to monitor a monitored piggery according to the first current state.

Optionally, the first current state determination module specifically includes:

a current food intake amount obtaining unit, configured to obtain a current food intake amount of the live pig within a period of time threshold range;

a first judgment unit, configured to determine whether the current food intake amount is in a downtrend within the time threshold range to obtain a first determining result;

a current environmental parameter obtaining unit, configured to obtain, if the first determining result indicates that the current food intake amount is in the downtrend within the time threshold range, current environmental parameters of the monitored piggery:

a second judgment unit, configured to determine whether the current environmental parameters and the historical environmental parameters are within an environmental-parameter similarity threshold range to obtain a second determining result; and

a first current state determination unit, configured to determine, if the second determining result indicates that the current environmental parameters and the historical environmental parameters are within the environmental-parameter similarity threshold range, by taking the current food intake amount and the current environmental parameters as an input, the first current state of the live pig according to the piggery monitoring model.

Optionally, the system for monitoring an epidemic disease in a piggery further includes:

a second current state obtaining module, configured to obtain a second current state of any live pig in the monitored piggery;

a third determining module, configured to determine whether the second current state and the first current state are within a first current-state similarity threshold range to obtain a third determining result;

a monitored piggery monitoring module, configured to determine, if the third determining result indicates that the second current state and the first current state are within the first current-state similarity threshold range, that no epidemic disease occurs in the monitored piggery; and

a first alarm module, configured to determine, if the third determining result indicates that the second current state and the first current state are not within the first current-state similarity threshold range, that an epidemic disease occurs in the monitored piggery and make an alarm to a farmer.

Optionally, the system for monitoring an epidemic disease in a piggery further includes:

a third current state obtaining module, configured to obtain a third current state of a live pig in any piggery;

a fourth determining module, configured to determine whether the third current state and the first current state are within a second current-state similarity threshold range to obtain a fourth determining result:

an any piggery monitoring module, configured to determine, if the third current state and the first current state are within the second current-state similarity threshold range, that the epidemic disease does not occur in the any piggery; and

a second alarm module, configured to determine, if the third current state and the first current state are not within the second current-state similarity threshold range, that the epidemic disease occurs in the any piggery and make an alarm to the farmer.

According to the specific embodiments provided by the present invention, the present invention discloses the following technical effects: with the adoption of the method for monitoring an epidemic disease in a piggery and system provided by the present invention, a piggery monitoring model is established, by taking the historical food intake amount and the historical environmental parameters as an input and the historical health state and the historical epidemic disease state as an output, according to a historical food intake amount, historical environmental parameters, a historical health state and a historical epidemic disease state of a live pig; and a current state of the live pig in a monitored piggery is monitored automatically according to the piggery monitoring model, thus determining whether the monitored piggery occurs an epidemic disease quickly and accurately; and meanwhile, the method can be applied to monitoring the epidemic disease throughout a country, so that before the epidemic disease breaks out, the epidemic disease can be detected timely and a reasonable countermeasure can be made.

### BRIEF DESCRIPTION OF THE DRAWINGS

To describe the technical solutions in the embodiments of the present invention or in the prior art more clearly, the following briefly introduces the accompanying drawings required for describing the embodiments. Apparently, the accompanying drawings in the following description show merely some embodiments of the present invention, and a person of ordinary skill in the art may still derive other drawings from these accompanying drawings without creative efforts.

FIG. 1 is a flow chart of a method for monitoring an epidemic disease in a piggery provided by the present invention; and

FIG. 2 is a structural diagram of a system for monitoring an epidemic disease in a piggery provided by the present invention.

### DETAILED DESCRIPTION OF THE ILLUSTRATED EMBODIMENTS

The following clearly and completely describes the technical solutions in the embodiments of the present invention with reference to the accompanying drawings in the embodiments of the present invention. Apparently, the described embodiments are merely a part rather than all of the embodiments of the present invention. All other embodiments obtained by a person of ordinary skill in the art based on the embodiments of the present invention without creative eflforts shall fall within the protection scope of the present invention.

An objective of the present invention is to provide a method for monitoring an epidemic disease in a piggery and system, which can improve the efficiency of monitoring an epidemic disease in a piggery.

To make the foregoing objective, features, and advantages of the present invention clearer and more comprehensible, the present invention is further described in detail below with reference to the accompanying drawings and specific embodiments.

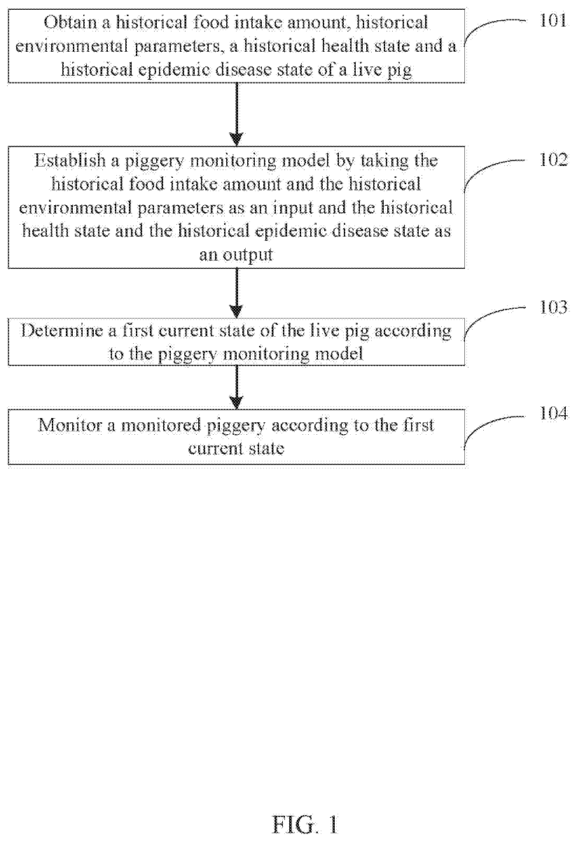

FIG. 1 is a flow chart of a method for monitoring an epidemic disease in a piggery provided by the present invention. As shown in FIG. 1, the method for monitoring an epidemic disease in a piggery includes the following steps.

Step 101: obtain a historical food intake amount, historical environmental parameters, a historical health state and a historical epidemic disease state of a live pig, where the historical environmental parameters include a temperature, a humidity, and concentration of gas in the air.

Step 102: establish a piggery monitoring model by taking the historical food intake amount and the historical environmental parameters as an input and the historical health state and the historical epidemic disease state as an output.

Step 103: determine a first current state of the live pig according to the piggery monitoring model, where the first current state includes a healthy state and an epidemic disease state.

The step 103 specifically includes: obtain a current food intake amount of the live pig within a period of time threshold range; determine whether the current food intake amount is in a downtrend within the period of time threshold range, and if the current food intake amount is in the downtrend, obtain current environmental parameters of the monitored piggery; determine whether the current environmental parameters and the historical environmental parameters are within an environmental-parameter similarity threshold range, and if yes, determine, by taking the current food intake amount and the current environmental parameters as an input, the first current state of the live pig according to the piggery monitoring model.

While a food intake amount of a group declines continuously, each environmental parameter in a piggery is normal, so it may be deduced that pigs are about to occur a disease; and after the group occurs the disease, symptoms from prediction of disease occurrence to the disease occurrence are recorded, e.g., the food intake amount declines quickly, the phenomenon that pigs die occurs continuously, etc.

When it is determined that the disease is not caused by a temperature, a humidity and an environmental problem, a piggery monitoring model under this environment is recorded and sorted out. When it is monitored that other piggeries also have the similar process, an analysis and a record may be made. When it is found that multiple piggeries within a range break out an epidemic disease condition suitable for this model in a same phase nearly, a nationwide early warning prompt is sent out in a platform system; and meanwhile, each piggery sends out an early warning prompt, so that a farmer makes preventive preparations in advance.

Step 104: monitor a monitored piggery according to the first current state.

After the step 104, the method further includes: obtain a second current state of any live pig in the monitored piggery; determine whether the second current state and the first current state are within a first current-state similarity threshold range; if yes, determine that no epidemic disease occurs in the monitored piggery; and if no, determine that an epidemic disease occurs in the monitored piggery and make an alarm to a farmer.

In response to determining that an epidemic disease occurs in the monitored piggery and making an alarm to a farmer, the method for monitoring an epidemic disease in a piggery further includes: obtain a third current state of a live pig in any piggery; determine whether the third current state and the first current state are within a second current-state similarity threshold range: if yes, determine that the epidemic disease does not occur in the any piggery; and if no, determine that the epidemic disease occurs in the any piggery and make an alarm to the farmer.

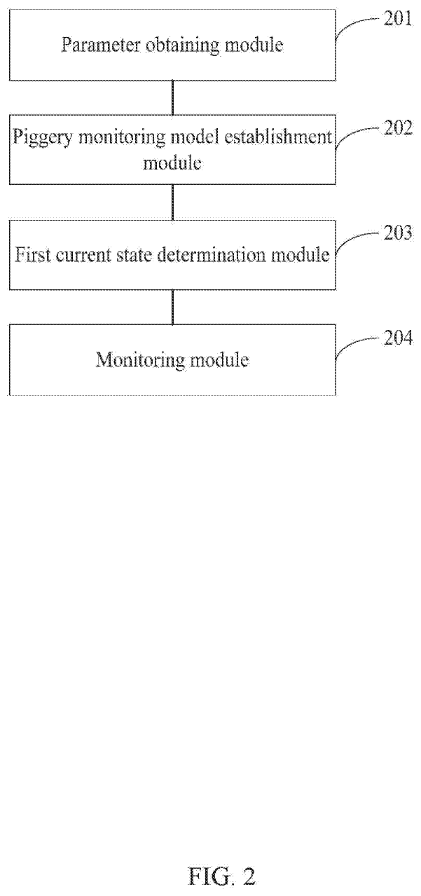

FIG. 2 is a structural diagram of a system for monitoring an epidemic disease in a piggery provided by the present invention. As shown in FIG. 2, the system for monitoring an epidemic disease in a piggery includes: a parameter obtaining module 201, a piggery monitoring model establishment module 202, a first current state determination module 203, and a monitoring module 204.

The parameter obtaining module 201 is configured to obtain a historical food intake amount, historical environmental parameters, a historical health state and a historical epidemic disease state of a live pig, where the historical environmental parameters include a temperature, a humidity, and concentration of gas in the air.

The piggery monitoring model establishment module 202 is configured to establish a piggery monitoring model by taking the historical food intake amount and the historical environmental parameters as an input and the historical health state and the historical epidemic disease state as an output.

The first current state determination module 203 is configured to determine a first current state of the live pig according to the piggery monitoring model, where the first current state includes a healthy state and an epidemic disease state.

The first current state determination module 203 specifically includes: a current food intake amount obtaining unit, configured to obtain a current food intake amount of the live pig within a period of time threshold range; a first judgment unit, configured to determine whether the current food intake amount is in a downtrend within the time threshold range to obtain a first determining result; a current environmental parameter obtaining unit, configured to obtain, if the first determining result indicates that the current food intake amount is in the downtrend within the time threshold range, current environmental parameters of the monitored piggery; a second judgment unit, configured to determine whether the current environmental parameters and the historical environmental parameters are within an environmental-parameter similarity threshold range to obtain a second determining result; and a first current state determination unit, configured to determine, if the second determining result indicates that the current environmental parameters and the historical environmental parameters are within the environmental-parameter similarity threshold range, by taking the current food intake amount and the current environmental parameters as an input, the first current state of the live pig according to the piggery monitoring model.

The monitoring module 204 is configured to monitor a monitored piggery according to the first current state.

The system for monitoring an epidemic disease in a piggery further includes: a second current state obtaining module, configured to obtain a second current state of any live pig in the monitored piggery; a third determining module, configured to determine whether the second current state and the first current state are within a first current-state similarity threshold range to obtain a third determining result; a monitored piggery monitoring module, configured to determine, if the third determining result indicates that the second current state and the first current state are within the first current-state similarity threshold range, that no epidemic disease occurs in the monitored piggery; and a first alarm module, configured to determine, if the third determining result indicates that the second current state and the first current state are not within the first current-state similarity threshold range, that an epidemic disease occurs in the monitored piggery and make an alarm to a farmer.

A third current state obtaining module is configured to obtain a third current state of a live pig in any piggery; a fourth determining module is configured to determine whether the third current state and the first current state are within a second current-state similarity threshold range to obtain a fourth determining result; an any piggery monitoring module is configured to determine, if the third current state and the first current state are within the second current-state similarity threshold range, that the epidemic disease does not occur in the any piggery; and a second alarm module is configured to determine, if the third current state and the first current state are not within the second current-state similarity threshold range, that the epidemic disease occurs in the any piggery and make an alarm to the farmer.

Each embodiment of the present specification is described in a progressive manner, each embodiment focuses on the difference from other embodiments, and the same and similar parts between the embodiments may refer to each other. For a system disclosed in the embodiments, since it corresponds to the method disclosed in the embodiments, the description is relatively simple, and reference can be made to the method description.

Several examples are used for illustration of the principles and implementation methods of the present invention. The description of the embodiments is used to help illustrate the method and its core principles of the present invention. In addition, those skilled in the art can make various modifications in terms of specific embodiments and scope of application in accordance with the teachings of the present invention. In conclusion, the content of this specification shall not be construed as a limitation to the invention.

What is claimed is:

1. A method for monitoring an epidemic disease in a piggery, comprising:
obtaining a historical food intake amount, historical environmental parameters, a historical health state and a historical epidemic disease state of a live pig, wherein the historical environmental parameters comprise a temperature, a humidity, and concentration of gas in the air;
establishing a piggery monitoring model by taking the historical food intake amount and the historical environmental parameters as an input and the historical health state and the historical epidemic disease state as an output;
determining a first current state of the live pig according to the piggery monitoring model, wherein the first current state comprises a healthy state and an epidemic disease state; and
monitoring a monitored piggery according to the first current state.

2. The method for monitoring an epidemic disease in a piggery according to claim 1, wherein the determining a first current state of the live pig according to the piggery monitoring model specifically comprises:
obtaining a current food intake amount of the live pig within a period of time threshold range;
determining whether the current food intake amount is in a downtrend within the time threshold range to obtain a first determining result;
if the first determining result indicates that the current food intake amount is in the downtrend within the time threshold range, obtaining current environmental parameters of the monitored piggery;
determining whether the current environmental parameters and the historical environmental parameters are within an environmental-parameter similarity threshold range to obtain a second determining result; and
if the second determining result indicates that the current environmental parameters and the historical environmental parameters are within the environmental-parameter similarity threshold range, determining, by taking the current food intake amount and the current environmental parameters as an input, the first current state of the live pig according to the piggery monitoring model.

3. The method for monitoring an epidemic disease in a piggery according to claim 1, after monitoring the monitored piggery according to the first current state, further comprising:
obtaining a second current state of any live pig in the monitored piggery;
determining whether the second current state and the first current state are within a first current-state similarity threshold range to obtain a third determining result;
if the third determining result indicates that the second current state and the first current state are within the first current-state similarity threshold range, determining that no epidemic disease occurs in the monitored piggery; and
if the third determining result indicates that the second current state and the first current state are not within the first current-state similarity threshold range, determining that an epidemic disease occurs in the monitored piggery and making an alann to a farmer.

4. The method for monitoring an epidemic disease in a piggery according to claim 3, after determining that the epidemic disease occurs in the monitored piggery and making the alarm to the farmer, further comprising:
obtaining a third current state of a live pig in any piggery;
determining whether the third current state and the first current state are within a second current-state similarity threshold range to obtain a fourth determining result;
if the third current state and the first current state are within the second current-state similarity threshold range, determining that the epidemic disease does not occur in the any piggery; and
if the third current state and the first current state are not within the second current-state similarity threshold range, determining that the epidemic disease occurs in the any piggery and making an alarm to the farmer.

5. A system for monitoring an epidemic disease in a piggery, comprising:
a parameter obtaining module, configured to obtain a historical food intake amount, historical environmental parameters, a historical health state and a historical epidemic disease state of a live pig, where the historical environmental parameters comprise a temperature, a humidity, and concentration of gas in the air;
a piggery monitoring model establishment module, configured to establish a piggery monitoring model by taking the historical food intake amount and the historical environmental parameters as an input and the historical health state and the historical epidemic disease state as an output;
a first current state determination module, configured to determine a first current state of the live pig according to the piggery monitoring model, where the first current state comprises a healthy state and an epidemic disease state; and
a monitoring module, configured to monitor a monitored piggery according to the first current state.

6. The system for monitoring an epidemic disease in a piggery according to claim 5, wherein the first current state determination module specifically comprises:
a current food intake amount obtaining unit, configured to obtain a current food intake amount of the live pig within a period of time threshold range;
a first judgment unit, configured to determine whether the current food intake amount is in a downtrend within the time threshold range to obtain a first determining result;
a current environmental parameter obtaining unit, configured to obtain, if the first determining result indicates that the current food intake amount is in the downtrend within the time threshold range, current environmental parameters of the monitored piggery;
a second judgment unit, configured to determine whether the current environmental parameters and the historical environmental parameters are within an environmental-parameter similarity threshold range to obtain a second determining result; and
a first current state determination unit, configured to determine, if the second determining result indicates that the current environmental parameters and the historical environmental parameters are within the environmental-parameter similarity threshold range, by taking the current food intake amount and the current environmental parameters as an input, the first current state of the live pig according to the piggery monitoring model.

7. The system for monitoring an epidemic disease in a piggery according to claim 5, further comprising:
a second current state obtaining module, configured to obtain a second current state of any live pig in the monitored piggery;
a third determining module, configured to determine whether the second current state and the first current state are within a first current-state similarity threshold range to obtain a third determining result;
a monitored piggery monitoring module, configured to determine, if the third determining result indicates that the second current state and the first current state are within the first current-state similarity threshold range, that no epidemic disease occurs in the monitored piggery; and
a first alarm module, configured to determine, if the third determining result indicates that the second current state and the first current state are not within the first current-state similarity threshold range, that an epidemic disease occurs in the monitored piggery and make an alarm to a farmer.

8. The system for monitoring an epidemic disease in a piggery according to claim 7, further comprising:
a third current state obtaining module, configured to obtain a third current state of a live pig in any piggery;
a fourth determining module, configured to determine whether the third current state and the first current state are within a second current-state similarity threshold range to obtain a fourth determining result;
an any piggery monitoring module, configured to determine, if the third current state and the first current state are within the second current-state similarity threshold range, that the epidemic disease does not occur in the any piggery; and
a second alarm module, configured to determine, if the third current state and the first current state are not within the second current-state similarity threshold range, that the epidemic disease occurs in the any piggery and make an alarm to the farmer.
